(19) United States
(12) Patent Application Publication
(10) Pub. No.: US 2006/0172967 A1
(43) Pub. Date: Aug. 3, 2006

(54) Method for producing alkyl-esterified glycosaminoglycan

(75) Inventor: Toshihiko Toida, Chiba-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Seikagaku Corporation, Chiyoda-ku (JP)

(21) Appl. No.: 11/242,896

(22) Filed: Oct. 5, 2005

(30) Foreign Application Priority Data
Jan. 31, 2005 (JP) 2005-024604

(51) Int. Cl. A61K 31/737; A61K 31/728; C08B 37/10; C08B 37/00; A61K 31/727

(52) U.S. Cl. 514/54; 514/56; 536/21; 536/53

(57) ABSTRACT

A method for producing an alkyl-esterified glycosaminoglycan, which comprises the step of allowing a trialkylsilyldiazoalkane to act on a glycosaminoglycan to perform alkyl-esterification of carboxyl groups of the glycosaminoglycan, and an alkyl-esterified glycosaminoglycan having a property that it is not substantially degraded by a glycosaminoglycan degrading enzyme such as hyaluronidase are provided.

This application claims the benefit of the convention priority based on Japanese Patent Application No. 2005-24604, filed on Jan. 31, 2005, the disclosure of which is incorporated herein by reference in its entirety.

### BACKGROUND OF THE INVENTION

The present invention relates to a method for producing an alkyl-esterified glycosaminoglycan and an alkyl-esterified glycosaminoglycan. More precisely, the present invention relates to a method for producing an alkyl-esterified glycosaminoglycan, which uses a trialkylsilyldiazoalkane, and an alkyl-esterified glycosaminoglycan.

Glycosaminoglycans are polysaccharides having a long chain structure constituted by repeating units of disaccharide consisting of an amino sugar and uronic acid or galactose, and are widely distributed mainly in connective tissues of animals. Examples of major glycosaminoglycans include hyaluronic acid, chondroitin, chondroitin sulfate, dermatan sulfate, heparin, heparan sulfate, keratan sulfate and so forth.

In recent years, physiological activities of these glycosaminoglycans attract attention. For example, hyaluronic acid is used as an ingredient of cosmetics and drugs. However, because glycosaminoglycans are ingredients of organisms, they are easily degraded in the living bodies, and have a problem that when they are used as a drug, they are degraded after administration and before they exert sufficient efficacy.

Therefore, it is being examined to inhibit degradation of glycosaminoglycans in the living bodies by modifying hydroxyl groups and carboxyl groups of glycosaminoglycans.

Examples of the method for modifying hydroxyl groups and carboxyl groups of glycosaminoglycans include alkyl-esterification, and for example, alkyl-esterified hyaluronic acid is known (for example, Japanese Patent No. 2569012).

Japanese Patent No. 2569012 describes a method of treating a quaternary ammonium salt of acidic polysaccharide having carboxyl groups with an esterifying agent (alkyl halide) in a neutral organic solvent as the only method of correctly controlling the number of carboxyl groups of acidic polysaccharide to be esterified. However, in this patent, any method of alkyl-esterifying a glycosaminoglycan without decomposing the glycosaminoglycan into glycosaminoglycans of lower molecular weights or method of producing an alkyl-esterified glycosaminoglycan comprising the step of allowing a trialkylsilyldiazoalkane to act on a glycosaminoglycan to alkyl-esterify carboxyl groups of the glycosaminoglycan is not described at all.

As a method for alkyl-esterification of glycosaminoglycans other than the methods described above, a method of alkyl-esterifying a saccharide using an alkylating agent such as diazomethane is also known (for example, Roger W. Jeanloz et al., J. Biol. Chem., Vol. 186, pp. 495-511, 1950). However, diazomethane is a substance having explosiveness and thus difficult in handling, and a method using a substance easier in handling is desirable.

Further, glycosaminoglycans may exhibit a molecular weight-specific physiological activity, and therefore a method that can be carried out under such a mild condition that the glycosaminoglycans should not be degraded into glycosaminoglycans of lower molecular weights during alkyl-esterification is desirable for obtaining a glycosaminoglycan of a specific molecular weight showing a specific physiological activity.

Although use of a trialkyldiazoalkane is known as an alkyl-esterification method of carboxylic acid (for example, Japanese Patent Unexamined Publication (KOKAI) No. 6-49089), no example of use of trialkyldiazoalkane for alkyl-esterification of glycosaminoglycan has been reported.

### SUMMARY OF THE INVENTION

Therefore, an object of the present invention is to provide a method for producing an alkyl-esterified glycosaminoglycan that can alkyl-esterify a glycosaminoglycan by using an alkyl-esterifying agent of which handling is easy, can alkyl-esterify carboxyl groups of a glycosaminoglycan to such a degree that degradation of the glycosaminoglycan in the living bodies can be inhibited to a desired degree, and can be carried out under such a condition that the glycosaminoglycan should not be degraded into lower molecular weight glycosaminoglycans during the alkyl-esterification, and to provide an alkyl-esterified glycosaminoglycan exhibiting degradation resistance to degradation by a glycosaminoglycan degrading enzyme.

The inventor of the present invention conducted various researches in order to achieve the aforementioned object, and as a result, he found that by allowing a trialkylsilyldiazoalkane, which has markedly superior handling property compared with diazomethane etc., to act on a glycosaminoglycan, the glycosaminoglycan could be sufficiently alkyl-esterified, and in addition, the reaction could be carried out under such a condition that the glycosaminoglycan should not be degraded into glycosaminoglycans of lower molecular weights during the alkyl-esterification. Furthermore, he also found that by allowing a trialkylsilyldiazoalkane to act on a glycosaminoglycan, substantially only carboxyl groups could be esterified without protecting hydroxyl groups etc. under such a condition that the glycosaminoglycan should not be degraded into glycosaminoglycans of lower molecular weights during the alkyl-esterification, and by controlling the reaction conditions etc., the esterification degree could be increased to such a degree that substantially all carboxyl groups could be esterified. The present invention was accomplished on the basis of these findings.

The present invention thus provides a method for producing an alkyl-esterified glycosaminoglycan, which comprises the step of allowing a trialkylsilyldiazoalkane to act on a glycosaminoglycan to perform alkyl-esterification of carboxyl groups of the glycosaminoglycan (henceforth also referred to as “the production method of the present invention”).

In this specification, the term “lower alkyl” or “lower alkane” means an alkyl or alkane having a straight or branched chain of 1 to 6 carbon atoms, unless particularly indicated.

The alkyl-esterification performed in the production method of the present invention is preferably (lower alkyl)-esterification, more preferably methyl-esterification.

In the production method of the present invention, the trialkylsilyldiazoalkane is preferably a trialkylsilyldiazo (lower alkane) having a silyl group such as trimethylsilyl group and t-butyldimethylsilyl group, more preferably a trimethylsilyldiazo (lower alkane), most preferably trimethylsilyldiazomethane.

In the production method of the present invention, the glycosaminoglycan is preferably selected from the group consisting of hyaluronic acid, chondroitin sulfate, chondroitin, dermatan sulfate, heparan sulfate and heparin, more preferably hyaluronic acid or chondroitin sulfate.

According to the production method of the present invention, substantially all carboxyl groups in a glycosaminoglycan can be alkyl-esterified.

The alkyl-esterified glycosaminoglycan produced by the production method of the present invention can be an alkyl-esterified glycosaminoglycan that is not degraded by one of glycosaminoglycan degrading enzymes including bovine testicular hyaluronidase, Streptomyces (Streptomyces hyalurolyticus) hyaluronidase, hyaluronidase SD and so forth, preferably by any of the foregoing exemplary glycosaminoglycan degrading enzymes, and more preferably, the alkyl-esterified glycosaminoglycan produced by the production method of the present invention can be an alkyl-esterified glycosaminoglycan that is not degraded by any of bovine testicular hyaluronidase, ovine testicular hyaluronidase, Streptomyces (Streptomyces hyalurolyticus) hyaluronidase and hyaluronidase SD, or by any of bovine testicular hyaluronidase, ovine testicular hyaluronidase, Streptomyces (Streptomyces hyalurolyticus) hyaluronidase, hyaluronidase SD and chondroitinase ABC.

The present invention also provides an alkyl-esterified glycosaminoglycan in which carboxyl groups are alkyl-esterified, which has a property that when a glycosaminoglycan degrading enzyme selected from the following (a) to (c) is allowed to act on the alkyl-esterified glycosaminoglycan under optimum conditions of the enzyme, the alkyl-esterified glycosaminoglycan is not substantially degraded:

(a) bovine testicular hyaluronidase

(b) hyaluronidase SD

(c) Streptomyces (Streptomyces hyalurolyticus) hyaluronidase (henceforth also referred to as “the alkyl-esterified glycosaminoglycan of the present invention”).

The alkyl-esterified glycosaminoglycan of the present invention is preferably a (lower alkyl)-esterified glycosaminoglycan, more preferably a methyl-esterified glycosaminoglycan.

In the alkyl-esterified glycosaminoglycan of the present invention, the glycosaminoglycan is preferably selected from the group consisting of hyaluronic acid, chondroitin sulfate, chondroitin, dermatan sulfate, heparan sulfate and heparin, more preferably hyaluronic acid or chondroitin sulfate.

The present invention further provides a composition for oral administration comprising the alkyl-esterified glycosaminoglycan of the present invention and a physiologically acceptable carrier, additive and/or auxiliary agent, wherein the alkyl-esterified glycosaminoglycan has resistance to degradation by a glycosaminoglycan degrading enzyme existing in living bodies (henceforth also referred to as “the composition for oral administration of the present invention”).

The present invention further provides the composition for oral administration of the present invention, which is a drug, cosmetic diet, functional food or health food.

The present invention further provides an injection comprising the alkyl-esterified glycosaminoglycan of the present invention and a physiologically acceptable carrier, additive and/or auxiliary agent, wherein the alkyl-esterified glycosaminoglycan has resistance to degradation by a glycosaminoglycan degrading enzyme existing in living bodies (henceforth referred to as “the injection of the present invention”).

Because the production method of the present invention uses a trialkylsilyldiazoalkane, which has markedly superior handling property compared with diazomethane etc., as an alkyl-esterifying agent, it can be easily performed, and it can also be sufficiently applicable to industrial production of alkyl-esterified glycosaminoglycan. Moreover, according to the production method of the present invention, by controlling the reaction conditions, the alkyl-esterification degree of the glycosaminoglycan can be controlled, and a desired degradation characteristic of the glycosaminoglycan in the living body can be obtained. In addition, the degree of the alkyl-esterification can be increased to such a degree that substantially all the carboxyl groups of the glycosaminoglycan should be alkyl-esterified, and thus the method increases alternatives of glycosaminoglycans that can be selected for the degradation characteristics thereof. Furthermore, because the alkyl-esterification reaction can be carried out under a milder condition according to the production method of the present invention compared with the alkyl-esterification reaction using hydrochloric acid and an alcohol, the alkyl-esterification reaction can be carried out so that the glycosaminoglycan should not be degraded into glycosaminoglycans of lower molecule weights during the reaction, and thus an alkyl-esterified glycosaminoglycan having a desired molecular weight can be easily produced. Moreover, according to the production method of the present invention, it is also possible to make only carboxyl groups esterified without protecting hydroxyl groups etc.

Although use of a trialkylsilyldiazoalkane as an alkyl-esterifying agent is known as described above, no example of use thereof for a glycosaminoglycan has been reported, and it has not been expected at all that use of a trialkylsilyldiazoalkane provides such advantages as described above.

### BRIEF DESCRIPTION OF THE DRAWINGS

FIG. 1 shows the results of NMR analysis of the methyl-esterified hyaluronic acid produced by the method of the present invention and the starting material hyaluronic acid.

FIG. 2 shows the results of two-dimensional NMR analysis of the methyl-esterified hyaluronic acid produced by the method of the present invention.

FIG. 3 shows the methyl-esterification rate of methyl-esterified hyaluronic acid.

FIG. 4 shows electropherograms of degradation products obtained from the methyl-esterified hyaluronic acid produced by the method of the present invention and the starting material hyaluronic acid by treatments with various enzymes.

FIG. 5 shows electropherograms of degradation products obtained from the methyl-esterified hyaluronic acid produced by the method of the present invention and the starting material hyaluronic acid by treatments with various enzymes.

FIG. 6 shows electropherograms of degradation products obtained from the methyl-esterified hyaluronic acid produced by the method of the present invention and the starting material hyaluronic acid by a treatment with chondroitinase ACII.

FIG. 7 shows the results of NMR analysis of degradation products obtained from the methyl-esterified hyaluronic acid produced by the method of the present invention and the starting material hyaluronic acid by a treatment with chondroitinase ACII.

FIG. 8 shows the results of NMR analysis of the methyl-esterified chondroitin sulfate produced by the method of the present invention and the starting material chondroitin sulfate.

### BEST MODE FOR CARRYING OUT THE INVENTION

1. Production Method of the Present Invention

Hereafter, the production method of the present invention will explained in detail.

The method for producing an alkyl-esterified glycosaminoglycan of the present invention is characterized by comprising the step of allowing a trialkylsilyldiazoalkane to act on a glycosaminoglycan.

Glycosaminoglycans, which are used as the starting material in the method for producing an alkyl-esterified glycosaminoglycan of the present invention, are polysaccharides having a long chain structure constituted by repeating units of disaccharide consisting of an amino sugar and uronic acid or galactose, and are widely distributed in connective tissues of animals and so forth.

Examples of the glycosaminoglycan usable in the production method of the present invention include hyaluronic acid, chondroitin sulfate, chondroitin, dermatan sulfate, heparin, heparan sulfate, N-acetyl heparosan and so forth. Preferred are hyaluronic acid, chondroitin sulfate, chondroitin, dermatan sulfate, heparan sulfate and heparin, more preferred are hyaluronic acid, chondroitin sulfate, chondroitin and dermatan sulfate, and the most preferred are hyaluronic acid and chondroitin sulfate.

The glycosaminoglycan is preferably in a form that can be dissolved or suspended in a reaction solvent, particularly preferably in a form of a free acid (free compound).

The origin of the glycosaminoglycan is not particularly limited, and for example, those isolated in a conventional manner from chicken crest, umbilical cord, skin, aorta and so forth of animals such as porcine, bovine, fish and other animals, microorganisms producing a glycosaminoglycan and so forth can be used.

Although the molecular weight of the glycosaminoglycan used for the production method of the present invention is not particularly limited, the molecular weight is generally about 1,000 to 5,000,000, preferably 10,000 to 2,000,000. When the glycosaminoglycan is hyaluronic acid, the average molecular weight thereof is preferably 1000 to 5,000,000, more preferably 10,000 to 2,000,000, most preferably 20,000 to 800,000. When the glycosaminoglycan is chondroitin sulfate, the average molecular weight thereof is preferably 1000 to 200,000, more preferably 10,000 to 100,000, most preferably 15,000 to 50,000. A glycosaminoglycan isolated from the materials mentioned above can be made into a lower molecule by subjecting it to a usual degradation treatment (e.g., enzymatic degradation, chemical degradation, heat treatment etc.) to obtain a glycosaminoglycan having a desired molecular weight.

In the method for producing an alkyl-esterified glycosaminoglycan of the present invention, a trialkylsilyldiazoalkane is used as an esterifying agent. The alkyl groups of the trialkyl moiety of the trialkylsilyldiazoalkane may be the same or different, and they are alkyl groups having preferably 1 to 4 carbon atoms, more preferably 1 to 2 carbon atoms, most preferably one carbon atom. The alkane moiety of the trialkylsilyldiazoalkane consists of an alkyl group having a straight or branched chain, and it is an alkane group having preferably 1 to 6 carbon atoms, more preferably 1 to 3 carbon atoms, most preferably one carbon atom.

The alkyl group of the alkyl ester moiety of the alkyl-esterified glycosaminoglycan produced by the production method of the present invention can be derived from the alkane moiety of the trialkylsilyldiazoalkane. Therefore, it is expected to be able to obtain an alkyl-esterified glycosaminoglycan having a specific carbon number within the alkyl-esterified moiety by selecting the carbon number of the alkane moiety of the trialkylsilyldiazoalkane.

Specific examples of the trialkylsilyldiazoalkane used for the production method of the present invention include trimethylsilyldiazoalkanes, in particular, trimethylsilyldiazomethane, trimethylsilyldiazoethane and so forth, and trimethylsilyldiazomethane is the most preferred. Therefore, the alkyl-esterification in the method for producing an alkyl-esterified glycosaminoglycan of the present invention is particularly preferably methyl-esterification.

As the trialkylsilyldiazoalkane used for the production method of the present invention, commercially available products can be used, and it can also be prepared by a known method (FIESER & FIESER, Reagents for Organic Synthesis, Vol. 4, 543).

The esterification reaction in the method for producing an alkyl-esterified glycosaminoglycan of the present invention can be carried out in a solvent.

The solvent used for the aforementioned reaction is not particularly limited so long as the solvent does not inhibit the reaction and can dissolve or suspend the starting materials to some extent. Examples thereof include, for example, alcohols such as methanol, ethanol, propanol, 2-propanol, butanol, isobutanol, t-butanol, pentanol and hexanol; aliphatic hydrocarbons such as hexane, heptane, ligroin and petroleum ether; aromatic hydrocarbons such as benzene, toluene and xylene; halogenated hydrocarbons such as methylene chloride, chloroform, carbon tetrachloride, dichloroethane, chlorobenzene and dichlorobenzene; ethers such as diethyl ether, diisopropyl ether, tetrahydrofuran, dioxane, dimethoxyethane and diethylene glycol dimethyl ether; esters such as methyl acetate and ethyl acetate; sulfoxides such as dimethyl sulfoxide (DMSO) and diethyl sulfoxide; N,N-dimethylformamide (DMF); mixed solvents of these solvents and so forth.

Among these, it is preferable to use a mixed solvent, more preferably a mixed solvent of a sulfoxide and an alcohol, most preferably a mixed solvent of dimethyl sulfoxide and methanol.

Although the procedure of the step of dissolving or suspending a glycosaminoglycan in a solvent is not particularly limited, when a mixed solvent of a sulfoxide and an alcohol is used, for example, it is preferable to dissolve the glycosaminoglycan in the sulfoxide and then add the alcohol to the solution. Further, when a mixed solvent of dimethyl sulfoxide and methanol is used, it is preferable to dissolve the glycosaminoglycan in dimethyl sulfoxide and then add methanol to the solution.

When a mixed solvent of a sulfoxide and an alcohol is used, the volume ratio of the sulfoxide and alcohol is preferably about 5:1 to 20:1, more preferably about 10:1.

Although the concentrations of the glycosaminoglycan and the trialkylsilyldiazoalkane in the reaction system are not particularly limited, if the concentrations (initial concentrations) of these substances are unduly high at the start of the reaction, precipitates containing the glycosaminoglycan and so forth may be unfavorably generated. It is generally preferred that the trialkylsilyldiazoalkane should be added in an amount more than the stoichiometric amount required for the desired esterification with respect to the glycosaminoglycan, and a higher ratio of the trialkylsilyldiazoalkane is more preferred in view of promotion of the reaction. However, an unduly high concentration of the trialkylsilyldiazoalkane is not preferred from the viewpoint described above. From these points of view, the concentration of the glycosaminoglycan in the reaction system is usually about 0.1 to 5 mg/ml, preferably about 0.5 to 3 mg/ml, more preferably about 1 mg/ml, the concentration of the trialkylsilyldiazoalkane is usually about 0.1 to 2% by weight, preferably about 0.2 to 1% by weight, more preferably about 0.7% by weight, and the ratio of the glycosaminoglycan and the trialkylsilyldiazoalkane is about 1:3 to 1:20, preferably about 1:5 to 1:10, more preferably about 1:6 to 1:8, in terms of weight ratio.

The other reaction conditions may also be selected from those used in usual esterification reactions using a trialkylsilyldiazoalkane. The reaction temperature is usually about 0 to 50° C., preferably about 10 to 40° C., more preferably about 15 to 30° C. The reaction time is usually about 10 minutes to 50 hours, preferably about 30 minutes to 10 hours, more preferably about 30 minutes to 3 hours.

In the method for producing an alkyl-esterified glycosaminoglycan of the present invention, by suitably adjusting the concentrations of reactants, reaction temperature, reaction time etc. as mentioned above for a specific starting material, the degree of the alkyl-esterification can be controlled to obtain a desired degree of the alkyl-esterification. As for preferred combination of the concentrations of reactants, reaction temperature and reaction time, the reaction is performed by using preferably about 0.5 to 3 mg/ml, more preferably about 1 mg/ml, of a glycosaminoglycan and preferably about 0.2 to 1% by weight, more preferably about 0.7% by weight, of a trialkylsilyldiazoalkane at a reaction temperature of preferably about 10 to 40° C., more preferably about 15 to 30° C., for preferably about 30 minutes to 10 hours, more preferably about 30 minutes to 3 hours.

In order to increase the degree of the alkyl-esterification, it is preferable, for example, to use a trialkylsilyldiazoalkane at a high concentration. However, use of a trialkylsilyldiazoalkane at a high concentration at the time of the start of the reaction has the aforementioned problem. It was found that, in such a case, a higher alkyl-esterification degree could be obtained by once performing the alkyl-esterification reaction under such reaction conditions (trialkylsilyldiazoalkane concentration etc.) that the aforementioned problem should not arise to attain a certain degree of alkyl-esterification, then adding a trialkylsilyldiazoalkane again to the solution after the reaction and further performing the reaction, or by once performing the alkyl-esterification reaction in a similar manner to attain a certain degree of alkyl-esterification, then purifying the alkyl-esterified glycosaminoglycan (purification can be performed by an appropriate combination of, for example, ethanol precipitation, dialysis and so forth) and further performing the alkyl-esterification in a similar manner for the purified glycosaminoglycan. Further, it was also found that, according to such a method, substantially all carboxyl groups in a glycosaminoglycan could be alkyl-esterified by using mild reaction conditions without causing the aforementioned problems such as precipitation in the reaction system.

Therefore, according to the method for producing an alkyl-esterified glycosaminoglycan of the present invention, any desired alkyl-esterification degree can be obtained by adjusting the concentrations of reactants, reaction temperature, reaction time, number of times of the reaction (once or multiple times of two or more times) and so forth.

Purification performed between the reactions when the aforementioned alkyl-esterification reaction is performed multiple times or purification of the final product can be performed by a known method. For example, it can be performed by centrifugation, precipitation, recrystallization, chromatography and so forth.

2. Alkyl-Esterified Glycosaminoglycan of the Present Invention

The alkyl-esterified glycosaminoglycan of the present invention is an alkyl-esterified glycosaminoglycan in which carboxyl groups are alkyl-esterified, which has a property that when a glycosaminoglycan degrading enzyme selected from the following (a) to (c) is allowed to act on the alkyl-esterified glycosaminoglycan under optimum conditions of the enzyme, the alkyl-esterified glycosaminoglycan is not substantially degraded:

(a) bovine testicular hyaluronidase

(b) hyaluronidase SD

(c) Streptomyces (Streptoomyces hyalurolyticus) hyaluronidase.

The alkyl-esterified glycosaminoglycan of the present invention can be preferably produced by the production method of the present invention. The meanings of the terms “alkyl-esterification”, “glycosaminoglycan”, and so forth are as those described in the explanation of the production method of the present invention. Further, the expression “to allow a glycosaminoglycan degrading enzyme to act under the optimum conditions of the enzyme” means that the reaction is performed by choosing reaction conditions optimum for the enzyme for reaction conditions including reaction temperature, reaction pH and so forth. These optimum reaction conditions can be easily chosen by those skilled in the art, and the reaction with such reaction conditions can be performed by the methods described in the examples contained in the present specification.

3. Composition for Oral Administration of the Present Invention

The composition for oral administration of the present invention is a composition for oral administration which contains the alkyl-esterified glycosaminoglycan of the present invention and a physiologically acceptable carrier, additive and/or auxiliary agent and wherein the alkyl-esterified glycosaminoglycan has degradation resistance to degradation by a glycosaminoglycan degrading enzyme existing in the living body.

The composition for oral administration of the present invention is an application of the alkyl-esterified glycosaminoglycan of the present invention as a composition for oral administration, and it can be made as a preparation suitable for oral administration using a physiologically acceptable carrier, additive and/or auxiliary agent.

Concentration and amount of the alkyl-esterified glycosaminoglycan of the present invention contained in the composition for oral administration of the present invention are not particularly limited, and they can be suitably chosen depending on purpose of administration, symptoms and age of patients or subjects as object of administration and so forth.

The composition for oral administration of the present invention may be in the form of, for example, a solid preparation such as powder, granule, capsule and tablet, or a liquid preparation. The composition may be in the form of drinkable preparation or food such as candy and jelly. Specific examples of the carrier, additive and auxiliary agent contained in the composition for oral administration of the present invention include saccharides, proteins, lipids, buffering agents, surfacants, colorants, preservatives and so forth, but they are not limited to these.

The composition for oral administration of the present invention can also be used as a component of drug, cosmetic diet, functional food or health food.

4. Injection of the Present Invention

The injection of the present invention is an injection which contains the alkyl-esterified glycosaminoglycan of the present invention and a physiologically acceptable carrier, additive and/or auxiliary agent and wherein the alkyl-esterified glycosaminoglycan has degradation resistance to degradation by a glycosaminoglycan degrading enzyme existing in the living body. The injection of the present invention is an application of the alkyl-esterified glycosaminoglycan of the present invention as an injection, and it can be made as a preparation suitable for injection with a physiologically acceptable carrier, additive and/or auxiliary agent.

Concentration and amount of the alkyl-esterified glycosaminoglycan of the present invention contained in the injection of the present invention are not particularly limited, and they can be suitably chosen depending on purpose of administration, symptoms and age of patients or subjects as object of administration and so forth.

Examples of the administration method of the injection of the present invention include intramuscular injection, subcutaneous injection, intradermal injection, intravenous injection and so forth, and it can be prepared as a suitable preparation depending on the administration method. Examples of the dosage form include solution, suspension, emulsion, solid formulation for dissolution before use, liposome preparation, gel and so forth. Specific examples of the carrier, additive and auxiliary agent used in the injection of the present invention include saccharides, proteins, lipids, distilled water for injection, buffers, surfactants, preservatives and so forth, but not limited to these.

### EXAMPLE

Hereafter, the present invention will be explained with reference to the following example. However, the present invention is not limited to the following example.

1. Preparation and Analysis of Methyl-Esterified Hyaluronic Acid 1

1-1. Preparation

Sodium hyaluronate (molecular weight: 20 kDa, Seikagaku Corporation) was made into free form by passing it thorough a Dowex 50 W-X8 column. The obtained free hyaluronic acid in an amount of 1 mg was dissolved in 1 mL of dimethyl sulfoxide (DMSO), 100 μL of methanol and 30 μL of trimethylsilyldiazomethane (Aldrich) were added to the solution, and the reaction was allowed at room temperature for 2 hours in the presence of nitrogen gas.

The reaction was terminated by adding 30 μL of acetic acid, and then 1 mL of water was added. Ethanol saturated with anhydrous sodium acetate in a volume 10 times the volume of the reaction solution was added to the reaction solution, and the mixture was left standing at 0° C. for 1 hour. Thereafter, the mixture was centrifuged in a centrifugation machine at 4° C. and 2500 g for 60 minutes to collect precipitates.

The precipitates were dissolved in 3 mL of water, to the solution was added 3 mL of ethyl acetate, and the mixture was stirred for 30 seconds. The solution was left standing, and then the separated aqueous layer (upper layer) was put into a dialysis tube and dialyzed against water. The dialyzed solution was lyophilized to obtain methyl-esterified hyaluronic acid.

1-2. NMR (Nuclear Magnetic Resonance) Analysis

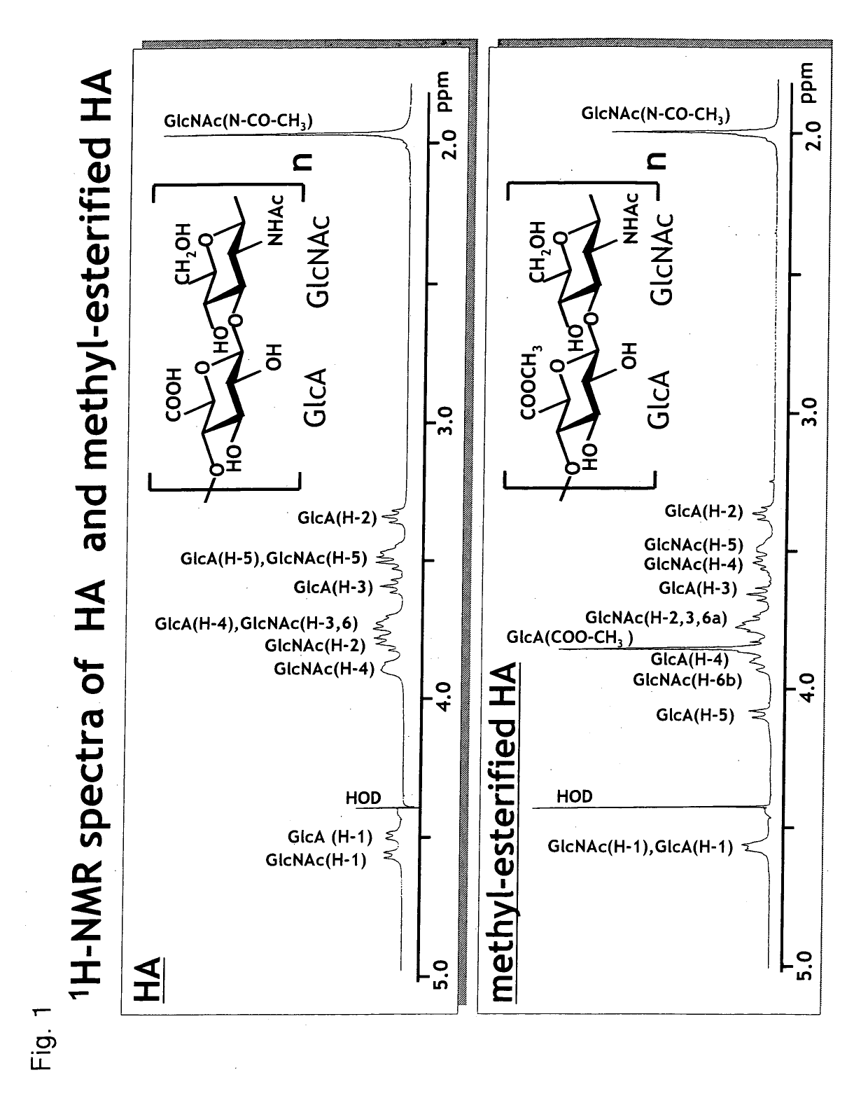

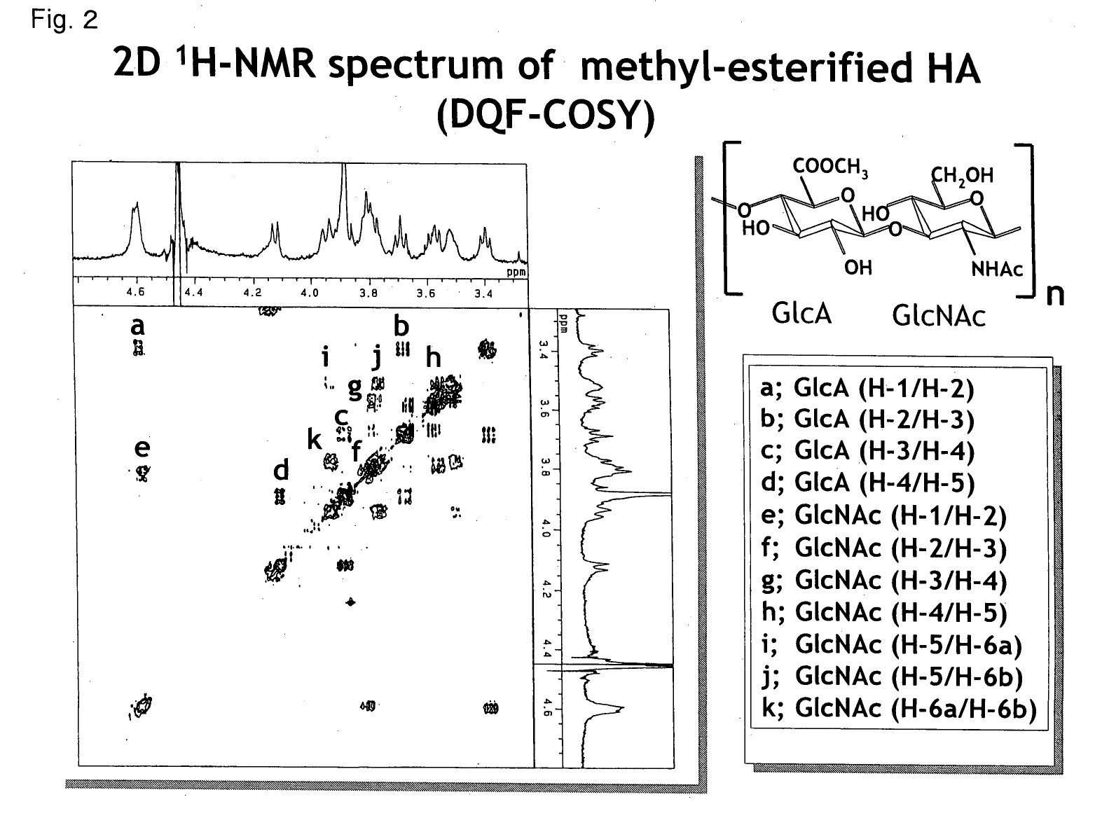

NMR analysis (¹H-NMR spectrum: δ (singlet of sodium trimethylsilylpropionate was used as a standard)) and two-dimensional NMR analysis of the sodium hyaluronate used above and the obtained methyl-esterified hyaluronic acid were performed. As the nuclear magnetic resonance measurement apparatus, GXSα500 (JEOL Co., Ltd) was used, and the measurement was performed in heavy water at room temperature. The results are shown in Table 1 and FIGS. 1 and 2.

TABLE 1
NMR data of sodium hyaluronate (HA) and methyl-esterified
hyaluronic acid (Methylated HA)

| | |
|---|---|
| HA | Methylated HA |

| | GlcA | GlcNAc | GlcA | GlcNAc |
|---|---|---|---|---|
| H-1 | 4.49 | 4.58 | 4.58 | 4.58 |
| H-2 | 3.37 | 3.80 | 3.37 | 3.76 |
| H-3 | 3.61 | 3.74 | 3.69 | 3.76 |
| H-4 | 3.75 | 3.88 | 3.82 | 3.54 |
| H-5 | 3.49 | 3.48 | 4.08 | 3.49 |
| H-6 | — | 3.73 | — | 3.74, 3.92 |
| N-acetyl | — | 1.98 | — | 2.02 |
| O-methyl | — | — | 3.82 | — |

From the results mentioned above, it was found that substantially only the carboxyl groups of the starting material hyaluronic acid is methyl-esterified.

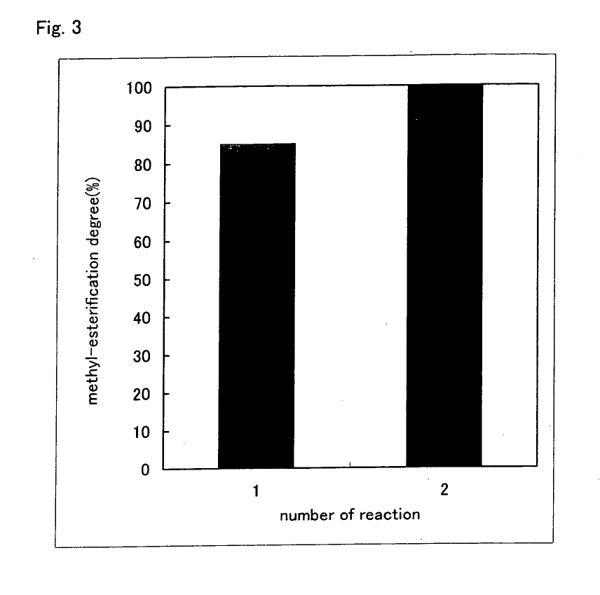

Further, the integral value of the peak originating in the introduced methyl group was analyzed to calculate methyl-esterification rate of the carboxyl groups of the glycosaminoglycan. The results are shown in FIG. 3 (the reaction was performed once). It was found that about 85% of the carboxyl groups were methyl-esterified as shown in FIG. 3.

1-3. Analysis of Molecular Weight

The molecular weights of the obtained methyl-esterified hyaluronic acid and the sodium hyaluronate as the starting material were measured. For the measurement of molecular weight, two of silica gel filtration columns (TSK gel SW3000, internal diameter: 8 mm, length: 50 cm) successively connected were used, and 100 mM phosphate buffer (pH 7.0) containing 0.2 M NaCl was used as an eluent. Ultraviolet absorbance was measured at 200 nm. A calibration curve was prepared by using hyaluronic acid molecular weight standards prepared beforehand by isolation from enzymatically partially degraded materials of macromolecular hyaluronic acid. By using this calibration curve and the elution time of the measurement samples, the molecular weights of the aforementioned methyl-esterified hyaluronic acid and sodium hyaluronate were calculated. The results are shown in Table 2.

Further, by using sodium hyaluronates having molecular weights of 130 kDa and 800 kDa (Seikagaku Corporation) instead of the sodium hyaluronate used as the starting material in 1-1, methyl-esterified hyaluronic acids were prepared in the same manner as in 1-1. Each of the starting material sodium hyaluronates and the obtained methyl-esterified hyaluronic acids was analyzed for the molecular weight in the same manner as described above (Table 2).

TABLE 2
Average molecular weight of sodium hyaluronate (HA) and
methyl-esterified hyaluronic acid (Methylated HA)

| HA | Methylated HA |
|---|---|
| 20,000 | 20,800 |
| 130,000 | 130,400 |
| 800,000 | 810,500 |

By the above experiment, it was confirmed that the molecular weights of the obtained methyl-esterified hyaluronic acids were not lowered compared with those of the starting material hyaluronic acids.

1-4. Enzymatic Degradation of Methyl-Esterified Hyaluronic Acids and Analysis of Degradation Products

In order to investigate degradation property of the methyl-esterified hyaluronic acid prepared above (1-1) with regard to degradation by hyaluronidase and chondroitinase, it was degraded with the conditions described below, and each of the degradation products were analyzed by capillary electrophoresis. For comparison, the sodium hyaluronate used as the starting material for the methyl-esterification was similarly degraded with each enzyme, and the degradation product was analyzed.

1) Enzymatic Degradation

The enzymes and reaction conditions used for the enzymatic degradation test are shown below.

(i) Bovine Testicular Hyaluronidase

Each of the prepared methyl-esterified hyaluronic acid and the starting material hyaluronic acid in an amount of 10 μg was dissolved in 1 mL of 50 mM phosphate buffer (pH 5.3), 1000 mU of bovine testicular hyaluronidase (Sigma) was added to the solution, and the reaction was allowed at 37° C. for 24 hours.

(ii) Ovine Testicular Hyaluronidase

Each of the prepared methyl-esterified hyaluronic acid and the starting material hyaluronic acid in an amount of 10 μg was dissolved in 1 mL of 50 mM phosphate buffer (pH 5.3), 1000 mU of ovine testicular hyaluronidase (Sigma) was added to the solution, and the reaction was allowed at 37° C. for 24 hours.

(iii) Streptomyces (Streptomyces hyalurolyticus) hyaluronidase

Each of the prepared methyl-esterified hyaluronic acid and the starting material hyaluronic acid in an amount of 10 μg was dissolved in 1 mL of 20 mM sodium acetate buffer (pH 6.0), 1 TRU of Streptomyces hyaluronidase (Amano Pharmaceuticals) was added to the solution, and the reaction was allowed at 55° C. for 24 hours.

(iv) Hyaluronidase SD

Each of the prepared methyl-esterified hyaluronic acid and the starting material hyaluronic acid in an amount of 10 μg was dissolved in 1 mL of 20 mM sodium acetate buffer (pH 6.0), 100 mU of hyaluronidase SD (Seikagaku Corporation) was added to the solution, and the reaction was allowed at 37° C. for 2 hours.

(v) Chondroitinase ABC

Each of the prepared methyl-esterified hyaluronic acid and the starting material hyaluronic acid in an amount of 10 μg was dissolved in 1 mL of 20 mM Tris-HCl buffer (pH 8.0), 100 mU of chondroitinase ABC (Seikagaku Corporation) was added to the solution, and the reaction was allowed at 37° C. for 2 hours.

(vi) Chondroitinase ACII

Each of the prepared methyl-esterified hyaluronic acid and the starting material hyaluronic acid in an amount of 10 μg was dissolved in 1 mL of 20 mM sodium acetate buffer (pH 6.0), 100 mU of chondroitinase ACII (Seikagaku Corporation) was added to the solution, and the reaction was allowed at 37° C. for 2 hours.

2) Capillary Electrophoresis

Each of the degradation products of the sodium hyaluronate and the methyl-esterified hyaluronic acid after the enzymatic degradation described above was analyzed by using a capillary electrophoresis apparatus (PACE5010, Beckman Coulter) equipped with a fused silica capillary column at 25° C. in the positive mode of 15 kV with sodium dodecyl sulfate/phosphate buffer (pH 8.0).

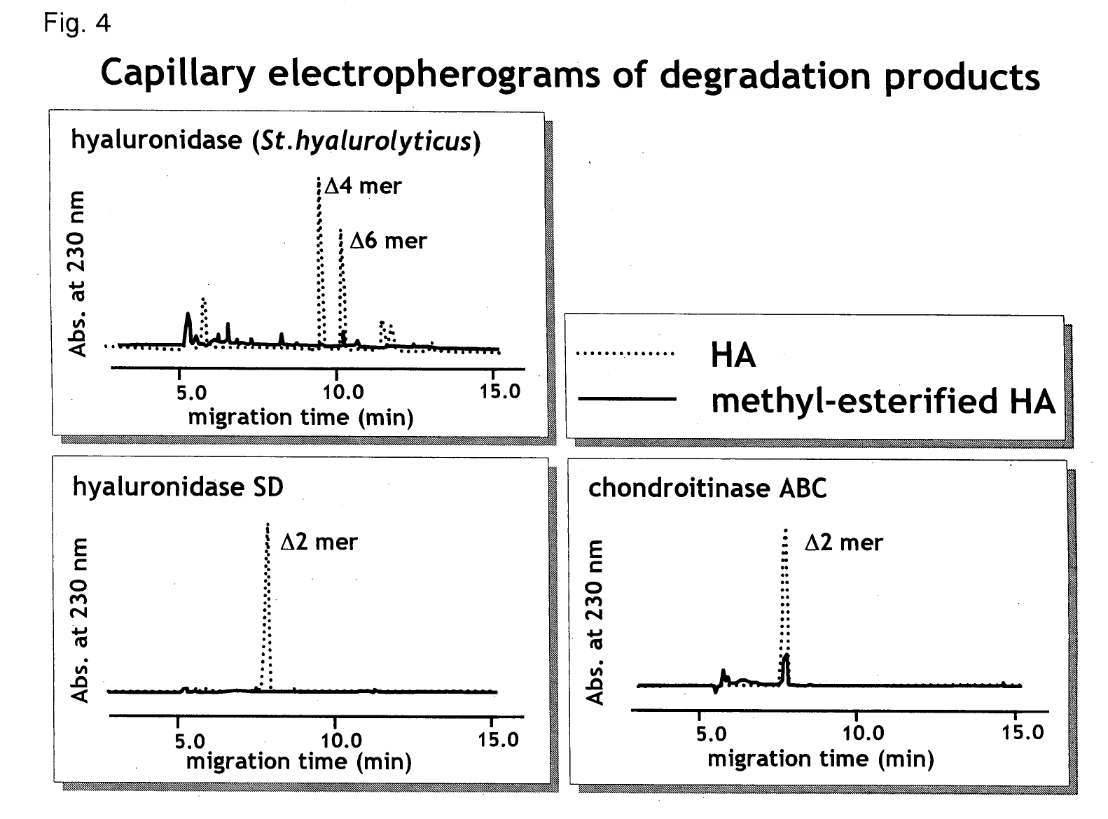

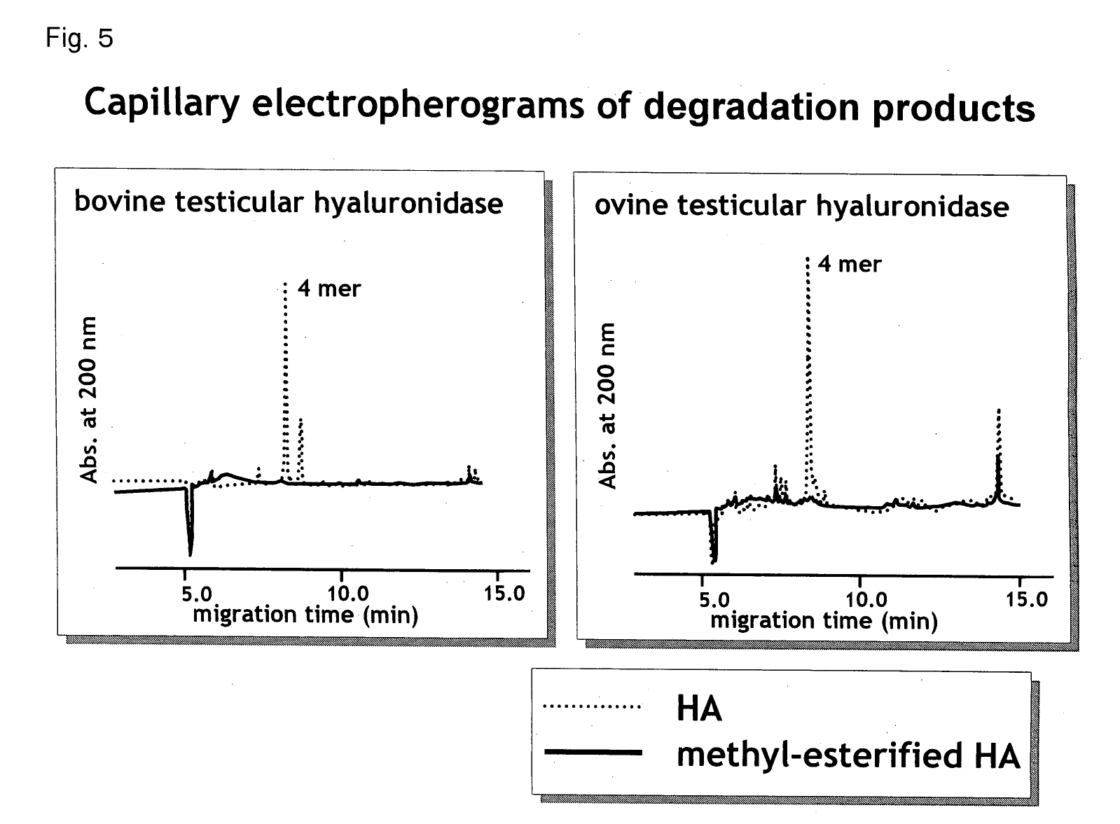

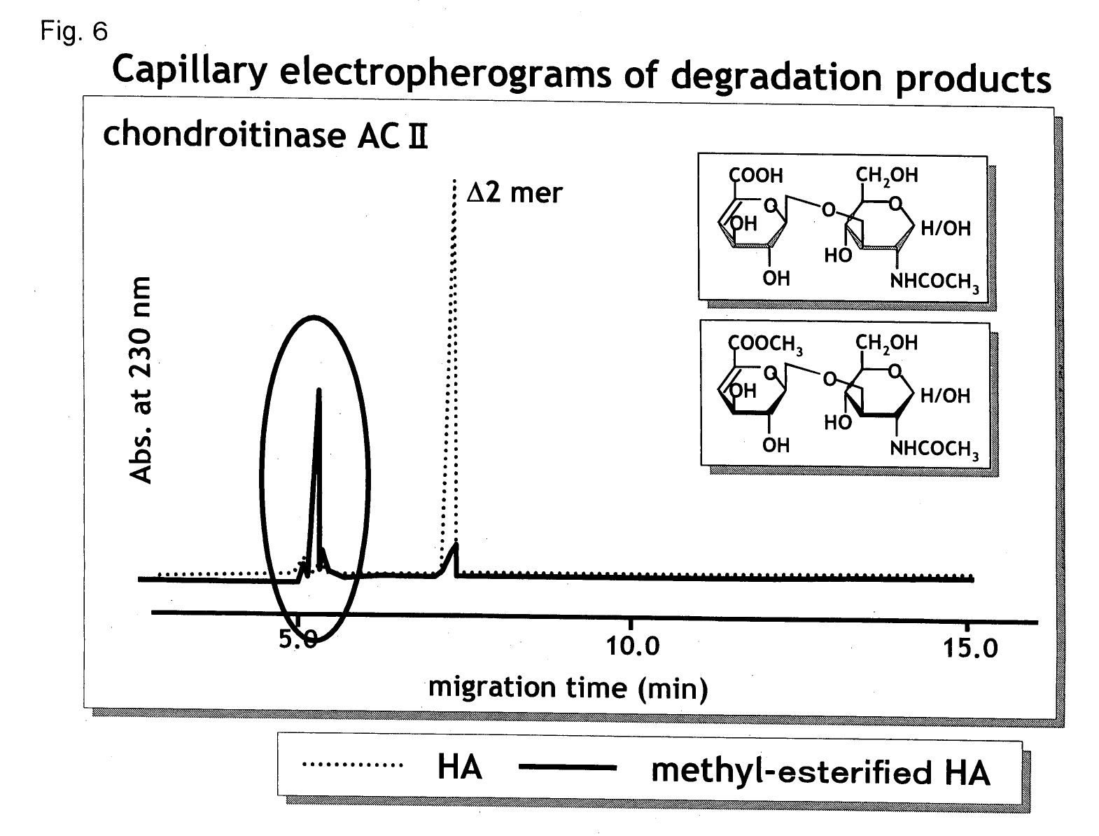

The results are shown in FIGS. 4 to 6. As clearly seen from the results, when the starting material hyaluronic acid was treated with each of the enzymes, peaks of tetrasaccharide (bovine testicular hyaluronidase, ovine testicular hyaluronidase), tetrasaccharide and hexasaccharide (Streptomyces (Streptomyces hyalurolyticus) hyaluronidase) and disaccharide (hyaluronidase SD, chondroitinase ABC) clearly appeared, and thus it was demonstrated that it was degraded by each of the enzymes. In contrast, when the methyl-esterified hyaluronic acid obtained by the method of the present invention was treated with each of the enzymes (bovine testicular hyaluronidase, ovine testicular hyaluronidase, Streptomyces (Streptomyces hyalurolyticus) hyaluronidase, hyaluronidase SD and chondroitinase ABC), substantially no peak corresponding to disaccharide, tetrasaccharide and hexasaccharide was observed, and thus it was demonstrated that the methyl-esterified hyaluronic acid was not substantially degraded by each of the enzymes. Further, when the starting material hyaluronic acid was treated with chondroitinase ACII, a peak of disaccharide clearly appeared. On the other hand, when the methyl-esterified hyaluronic acid obtained by the method of the present invention was treated with chondroitinase ACII, a definite peak was confirmed at a position different from that of the aforementioned peak of disaccharide (FIG. 6, indicated with a circle).

3) NMR Analysis of Enzymatic Degradation Products

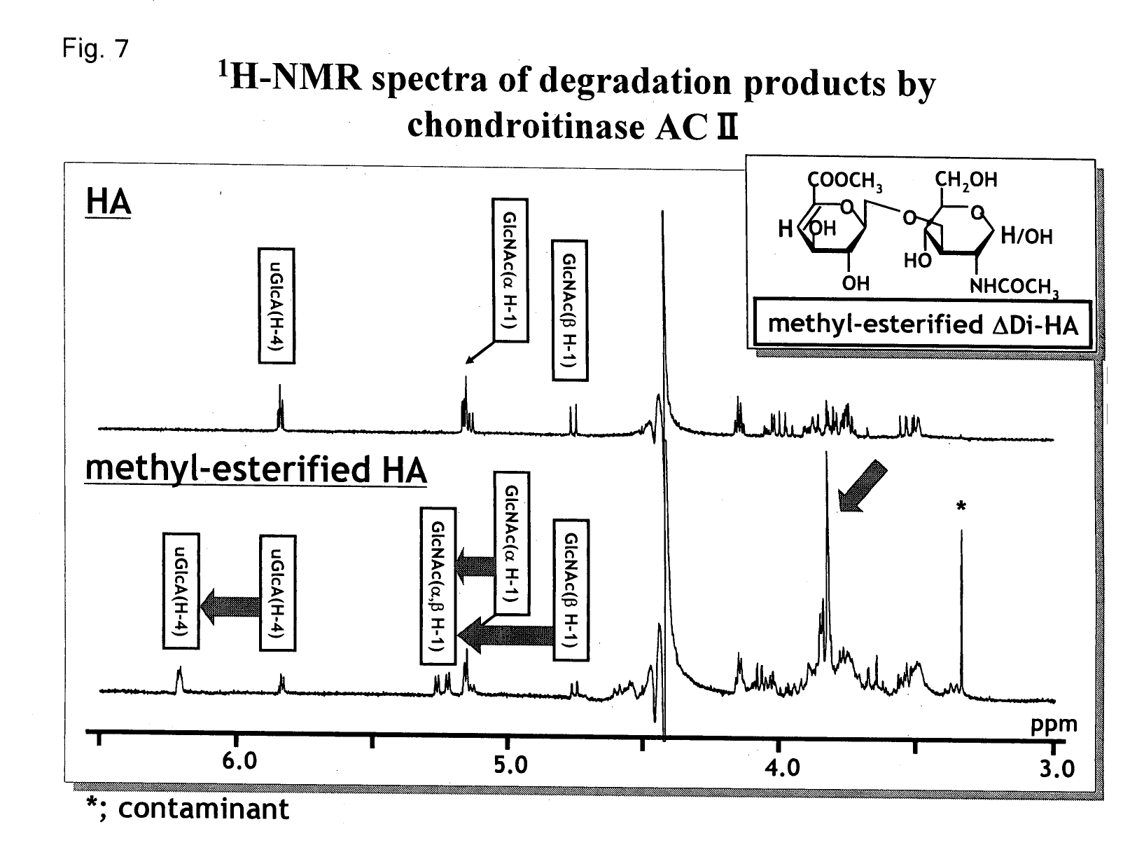

NMR analysis was performed for the methyl-esterified hyaluronic acid and the starting material hyaluronic acid after they were subjected to a treatment with chondroitinase ACII mentioned above. The results are shown in FIG. 7. As shown by the results, presence of methyl ester group was confirmed for the methyl-esterified hyaluronic acid degradation product (FIG. 7, indicated with an arrow on the right side in the spectrum of the methyl-esterified HA), which was not seen for the degradation product of the starting material hyaluronic acid. Moreover, the glucuronic acid H-5 at the root of the carboxyl group changed due to the methyl-esterification of the carboxyl group, and N-acetylglucosamine H-1 changed due to change of magnetic anisotropy. Further, in FIG. 7, the shifts of the signals of N-acetylglucosamine H-1 and glucuronic acid H-4 due to the methyl-esterification are indicated with horizontal arrows. From the above, it was confirmed that the methyl-esterified hyaluronic acid was degraded by chondroitinase ACII to produce a methyl-esterified disaccharide.

2. Preparation and Analysis of Methyl-Esterified Hyaluronic Acid 2

A methyl-esterified hyaluronic acid was obtained in the same manner as in the preparation and analysis of methyl-esterified hyaluronic acid 1 (1-1). By using the obtained methyl-esterified hyaluronic acid as a starting material, methyl-esterification and purification were performed again in the same manner as in the preparation and analysis of methyl-esterified hyaluronic acid 1. It was confirmed that, in the obtained methyl-esterified hyaluronic acid, substantially all the carboxyl groups were methyl-esterified (FIG. 3, the reaction was performed 2 times).

3. Preparation and Analysis of Methyl-Esterified Hyaluronic Acid 3

Methyl-esterification of hyaluronic acid was performed in the same manner as in the preparation and analysis of methyl-esterified hyaluronic acid 1 (1-1) except that the reaction time after the addition of trimethylsilyldiazomethane was changed to 1 hour. After completion of the reaction, the same amount of trimethylsilyldiazomethanes was added again, and the reaction was similarly allowed for 1 hour. In the same manner as in the preparation and analysis of methyl-esterified hyaluronic acid 1, the reaction was terminated, and the methyl-esterified hyaluronic acid was purified. It was confirmed in the same manner as in 1-2 that, in the obtained methyl-esterified hyaluronic acid, substantially all the carboxyl groups were methyl-esterified.

4. Preparation and Analysis of Methyl-Esterified Chondroitin Sulfate, Methyl-Esterified Chondroitin, Methyl-Esterified Dermatan Sulfate, Methyl-Esterified Heparan Sulfate And Methyl-Esterified Heparin

In the same manner as in 1-1 (preparation of methyl-esterified hyaluronic acid) except that sodium chondroitin sulfate (molecular weight: 15 kDa, 30 kDa, 42 kDa), chondroitin (molecular weight: 10 kDa), dermatan sulfate (molecular weight: 40 kDa), heparan sulfate (molecular weight: 30 kDa) and heparin (molecular weight: 30 kDa) (these various glycosaminoglycans were produced by Celsus, Ohio, U.S.) were used instead of the starting material used in 1-1, methyl-esterified chondroitin sulfate, methyl-esterified chondroitin, methyl-esterified dermatan sulfate, methyl-esterified heparan sulfate and methyl-esterified heparin were prepared.

5. Analysis of Methyl-Esterified Chondroitin Sulfate, Methyl-Esterified Chondroitin and Methyl-Esterified Dermatan Sulfate

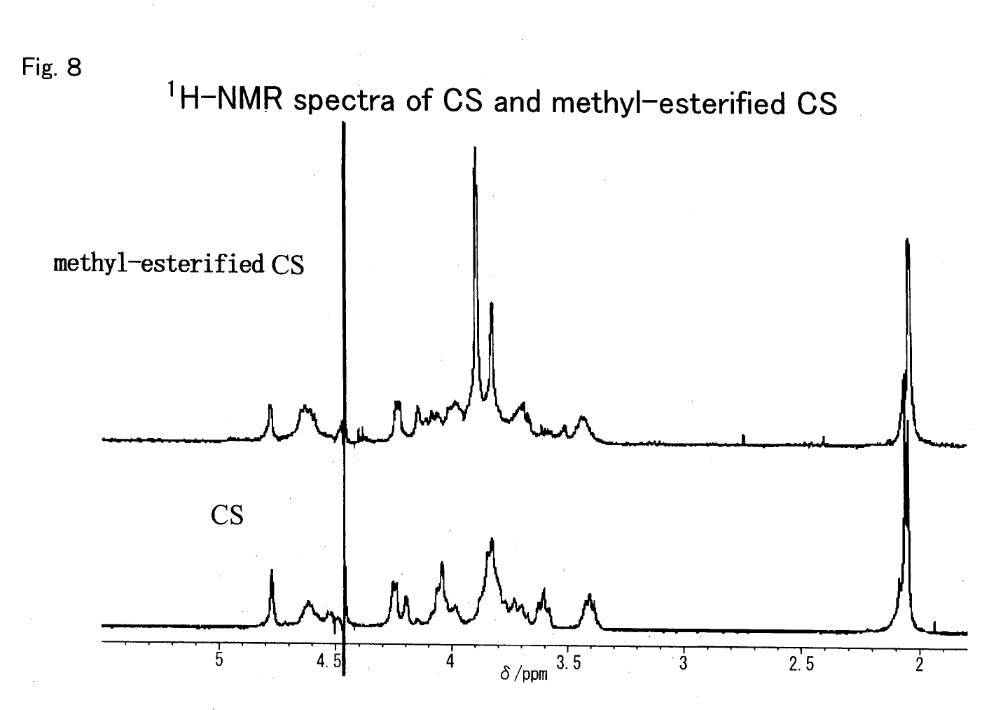

By NMR analysis performed in the same manner as that used for methyl-esterified hyaluronic acid (see 1-2), it was confirmed that only carboxyl groups of the chondroitin sulfate, chondroitin and dermatan sulfate were methyl-esterified. As an example, the ¹H-NMR spectra of the chondroitin sulfate (CS) and methyl-esterified chondroitin sulfate are shown in FIG. 8. It was confirmed that the signal of GalNAc6S H-6 around 4.8 ppm became smaller due to the methylation. Moreover, it was confirmed that a large singlet originating in methyl ester appeared around 3.9 ppm, and the intensity of the peak was almost equal to that of the peak originating in N-acetylmethyl. Further, the molecular weights of the methyl-esterified chondroitin sulfates and the chondroitin sulfates used as the starting material were analyzed in the same manner as in 1-3. The results are shown in Table 3 below. It was confirmed that the molecular weights of the obtained methyl-esterified chondroitin sulfates were not lowered compared with those of the starting material chondroitin sulfates.

TABLE 3
Average molecular weight of chondoroitin sulfate (CS) and
methyl-esterified chondoroitin sulfate (Methylated CS)

| CS | Methylated CS |
|---|---|
| 15,000 | 15,400 |
| 30,000 | 30,500 |
| 42,000 | 42,200 |

According to the method for producing an alkyl-esterified glycosaminoglycan of the present invention, a glycosaminoglycan can be esterified by using a trialkylsilyldiazoalkane, of which handling property is markedly superior to that of diazomethane etc., under milder conditions compared with those required for the alkyl-esterification using hydrochloric acid and an alcohol, and thus it is also extremely useful for industrial production of alkyl-esterified glycosaminoglycans. Moreover, by controlling the reaction conditions etc. in the method, degree of the alkyl-esterification of the glycosaminoglycan can be controlled in a wide range, and the method does not lower the molecular weight of the starting material glycosaminoglycan. Therefore, the method is extremely useful for production of drugs utilizing glycosaminoglycans and so forth.

What is claimed is:

1. A method for producing an alkyl-esterified glycosaminoglycan, which comprises the step of allowing a trialkylsilyldiazoalkane to act on a glycosaminoglycan to perform alkyl-esterification of carboxyl groups of the glycosaminoglycan.

2. The method according to claim 1, wherein the alkyl-esterification is lower alkyl-esterification.

3. The method according to claim 2, wherein the lower alkyl-esterification is methyl-esterification.

4. The method according to claim 1, wherein the trialkylsilyldiazoalkane is a trimethylsilyldiazo (lower alkane).

5. The method according to claim 4, wherein the trialkylsilyldiazo (lower alkane) is trimethylsilyldiazomethane.

6. The method according to claim 1, wherein the glycosaminoglycan is selected from the group consisting of hyaluronic acid, chondroitin sulfate, chondroitin, dermatan sulfate, heparan sulfate and heparin.

7. The method according to claim 6, wherein the glycosaminoglycan is hyaluronic acid or chondroitin sulfate.

8. The method according to claim 1, wherein substantially all carboxyl groups in the alkyl-esterified glycosaminoglycan are alkyl-esterified.

9. The method according to claim 1, wherein the alkyl-esterified glycosaminoglycan is not substantially degraded by at least one of bovine testicular hyaluronidase, Streptomyces (Streptomyces hyalurolyticus) hyaluronidase and hyaluronidase SD.

10. An alkyl-esterified glycosaminoglycan in which carboxyl groups are alkyl-esterified, which has a property that when a glycosaminoglycan degrading enzyme selected from the following (a) to (c) is allowed to act on the alkyl-esterified glycosaminoglycan under optimum conditions of the enzyme, the alkyl-esterified glycosaminoglycan is not substantially degraded:
(a) bovine testicular hyaluronidase
(b) hyaluronidase SD
(c) Streptomyces (Streptomyces hyalurolyticus) hyaluronidase.

11. The alkyl-esterified glycosaminoglycan according to claim 10, which is a (lower alkyl)-esterified glycosaminoglycan.

12. The alkyl-esterified glycosaminoglycan according to claim 11, which is a methyl-esterified glycosaminoglycan.

13. The alkyl-esterified glycosaminoglycan according to claim 10, wherein the glycosaminoglycan is selected from the group consisting of hyaluronic acid, chondroitin sulfate, chondroitin, dermatan sulfate, heparan sulfate and heparin.

14. The alkyl-esterified glycosaminoglycan according to claim 13, wherein the glycosaminoglycan is hyaluronic acid or chondroitin sulfate.

15. The alkyl-esterified glycosaminoglycan according to claim 10, wherein substantially all carboxyl groups in the alkyl-esterified glycosaminoglycan are alkyl-esterified.
